(19) United States
(12) Patent Application Publication
(10) Pub. No.: US 2012/0116245 A1
(43) Pub. Date: May 10, 2012

(54) PUNCTURE DEVICE

(76) Inventor: Hans-Peter Steiner, Graz (AT)

(21) Appl. No.: 13/380,596

(22) PCT Filed: Jun. 18, 2010
(86) PCT No.: PCT/EP10/58623

§ 371 Date: Jan. 24, 2012

(30) Foreign Application Priority Data
Jun. 25, 2009 (AT) A 980/2009

(51) Int. Cl. A61B 10/02

(52) U.S. Cl. 600/563; 600/567; 600/566

(57) ABSTRACT

A puncturing device for extracting egg cells for in-vitro fertilization (IVF) includes a puncturing cannula that can be connected by means of a multiway valve to a suction line or a flushing line. The multiway valve is provided with a foot-operated, mechanical or electro-motive drive mechanism for switching the multiway valve from a suction position to a flushing position and vice versa.

The invention relates to a puncturing device, preferably for extracting egg cells for in-vitro fertilization (IVF), with a puncturing cannula that can be connected to a suction line or to a flushing line by means of a multiway valve.

In the context of infecundity therapy using classical in-vitro fertilization, egg cells are obtained by puncturing the follicle immediately prior to expected ovulation. Maturing of the egg cells is assisted by means of hormone treatment for approximately ten days, which will result in the simultaneous maturing of several egg cells and thus in a higher success rate of IVF. Follicle puncture is carried out with ultrasonic guidance. The aim is to obtain egg cells from all follicles of more than 10 mm size to optimize the pregnancy rate.

The finding rate for egg cells without flushing the follicles with flushing fluid is 60% to 70%. To increase this rate double-lumen needles have been used since the middle of the nineteen eighties for flushing follicles (one lumen for flushing, one lumen for aspiration). Such needles have the disadvantage of a large exterior diameter, however, making the procedure more painful for the patient and requiring increased administering of analgesics.

It is furthermore known (see e.g. AT 505.749 A4) to use puncturing devices with a flushing component, where by means of a three-way valve connected to a thin single-lumen puncture needle, flushing or aspiration may be alternatively effected depending on the position of the three-way valve. The single-lumen needle is alternatingly connected via the three-way valve to flushing tubing or to aspiration or suction tubing. After the follicle has undergone aspiration the valve is switched, connecting the needle to a flushing syringe. Under direction of the surgeon the syringe is activated either manually by a helper or by means of an electric motor and the follicle is filled again. If the three-way valve is now switched back to the initial position by the surgeon, the redistended follicle is again emptied by activating the vacuum pump.

From U.S. Pat. No. 4,731,052 A there is known a puncturing device for the extraction of egg cells, having a puncturing cannula which may be connected to a flexible suction line and a flushing line. The device is not furnished with a three-way valve which can connect either to a suction or a flushing line, but only has a stop valve with a spring-loaded key, which in the inactive position disconnects the flexible suction line and opens the suction line when the key is manually pressed. U.S. Pat. No. 4,731,052 A provides a separate valve for the flushing line, thus necessitating considerable manipulative effort in the use of this known device.

In CN 201223430 Y there is described a puncturing device with a puncturing cannula, which can be optionally connected to a suction line or a flushing line by means of a multiway valve.

Finally there is known from DE 36 30 203 A1 a device for extracting tissue, which is provided with a control cable, where a driving mechanism for the cable can be activated by a foot-operated switch. A cutting mechanism comprising an inner pipe slideable in a fixed outer pipe, is controlled by means of the cable, and tissue is cut by cutting edges and received in a hollow space. The suction process is controlled by a simple magnetic valve placed in the suction line.

It is a disadvantage of the conventional procedure that the surgeon not only has to manipulate the puncturing cannula and the ultrasound probe independently of each other, but must also carry out a number of switching processes from flushing to suction operation, which usually requires the assistance of a helper. Many surgeons therefore avoid the stressful and inconvenient flushing operation and thus forego a higher yield of egg cells with the result of a lower pregnancy rate.

Based on the known puncturing devices for in-vitro fertilization initially described, it is the object of the present invention to propose improvements which will enable the surgeon to work without stress and without needing a helper.

The invention achieves this object by proposing that the multiway valve be provided with a foot-operated, mechanical or electrical drive unit for switching the valve from suction position to flushing position and vice versa.

In particular, the invention proposes that the drive unit have a grip element or handpiece comprising an actuator part, which can be attached mechanically to the multiway valve carrying the puncturing cannula, for instance by a locking or snap-on connection.

According to a first variant of the invention the actuator part for the multiway valve is force-connected via a control cable to an actuator element in a foot-operated rocker. The puncturing device of the invention thus comprises a 15 cm handpiece or grip-shaped housing gripped by the surgeon with one hand, onto whose mechanism the multiway valve is snap-connected, the valve being directly attached to the Luer connector of the preferably single-lumen puncturing needle. The ultrasound probe may be independently guided by the other hand, all switching operations of the multiway valve being carried out by the foot-operated rocker switch.

According to the invention the handpiece may be configured in two parts; a first part which holds the actuator part and the toothed rod and a second part which is inserted into the first part and receives the end of the control cable. The two parts of the handpiece are joined by means of a spring-loaded locking element, thus permitting the handpiece to be disassembled without the use of tools and detached from the control cable and sterilized.

According to a second variant of the invention the actuator part for the multiway valve may be driven by a gear motor placed in the handpiece, which is connected to a switch in the foot-operated rocker. In this variant, too, all switching operations of the multiway valve maybe carried out by the foot-operated rocker.

The invention will now be described in more detail with reference to the enclosed drawings. There is shown in

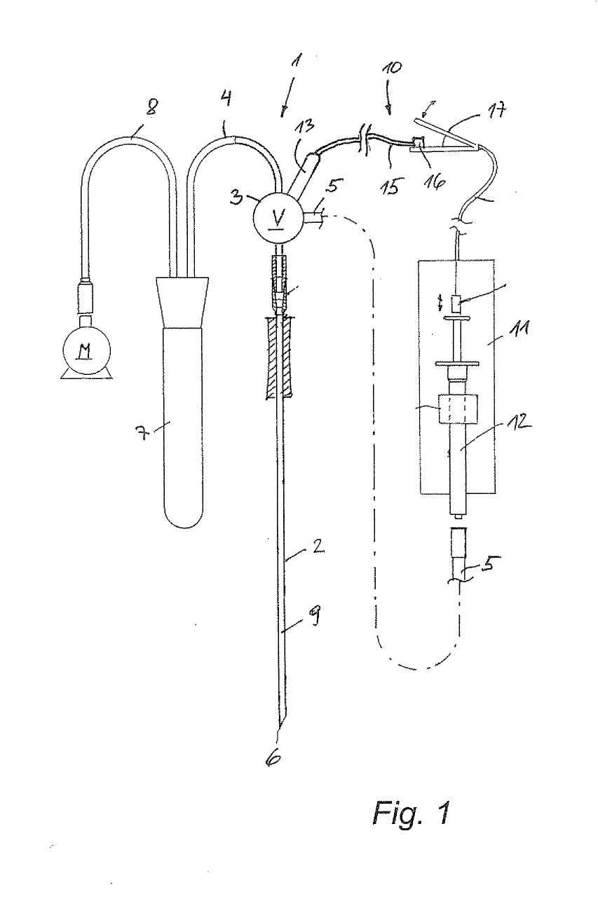

FIG. 1 a first variant of a puncturing device for the extraction of egg cells according to the invention in a schematic overall view;

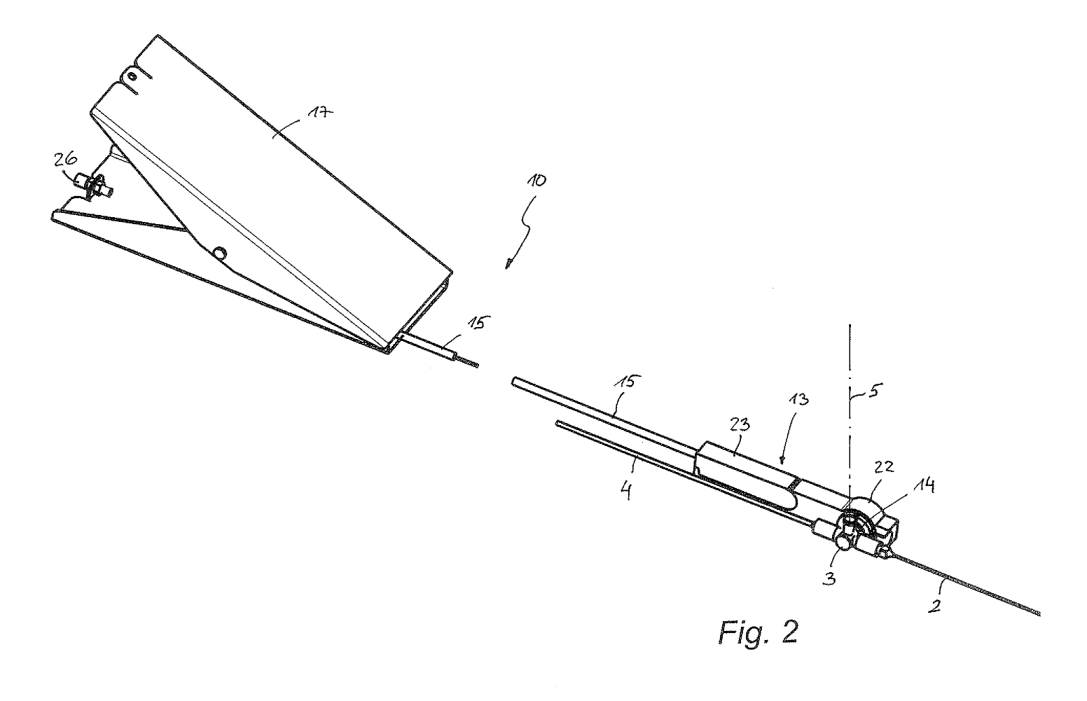

FIG. 2 a first variant of the drive mechanism of the multiway valve of the puncturing device in a three-dimensional view;

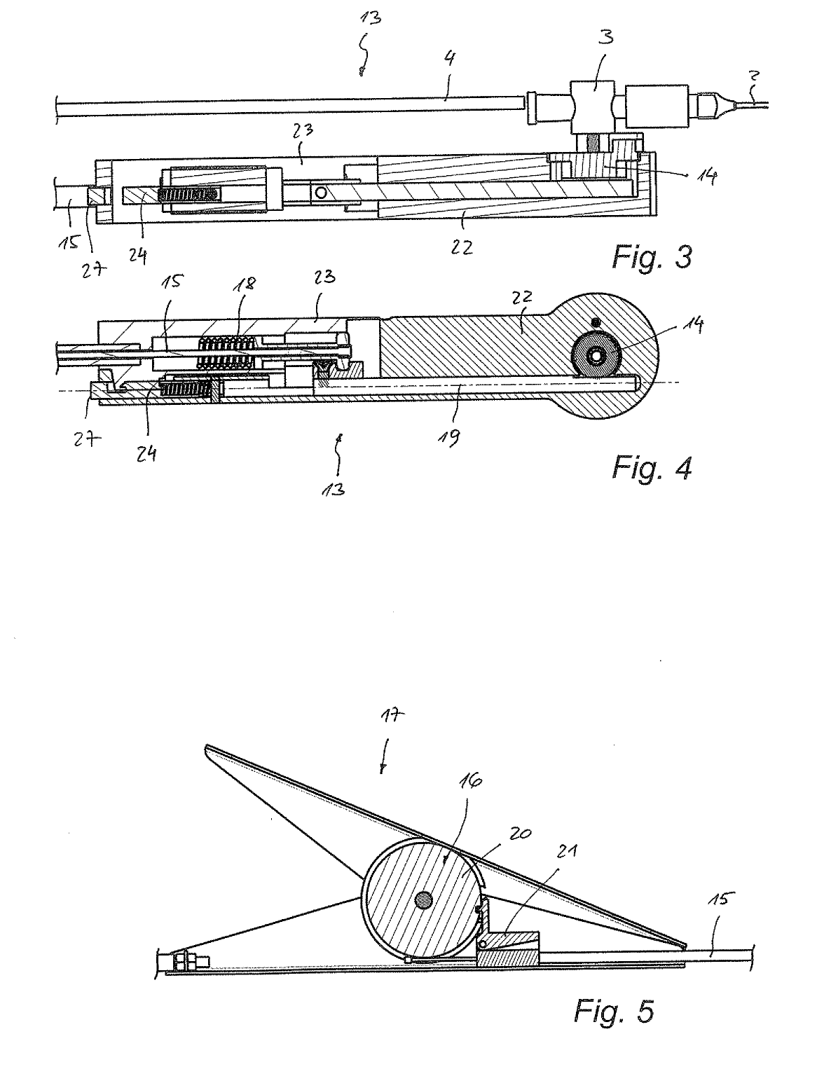

FIG. 3 and FIG. 4 sectional views of the handpiece of the drive mechanism according to FIG. 2;

FIG. 5 a sectional view of the foot-operated rocker according to FIG. 2;

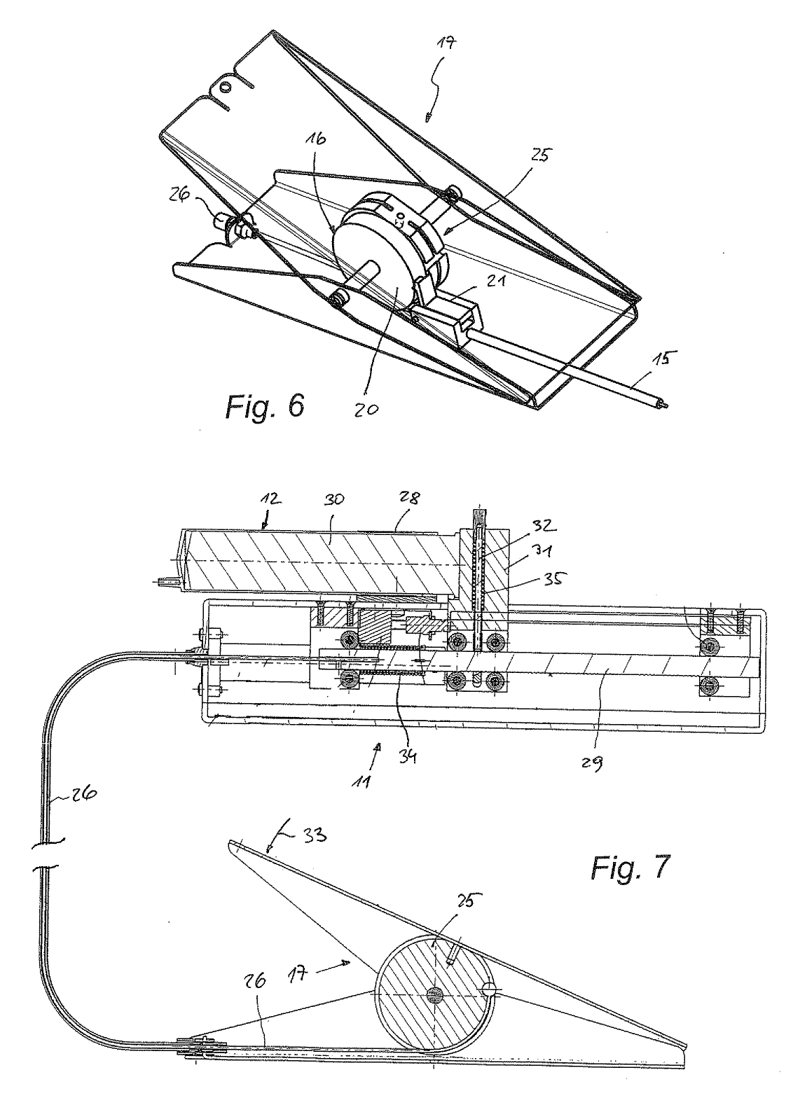

FIG. 6 a three-dimensional view of the foot-operated rocker of FIG. 5;

FIG. 7 a foot-operated rocker according to FIG. 6 for actuating a plunger syringe containing the flushing medium;

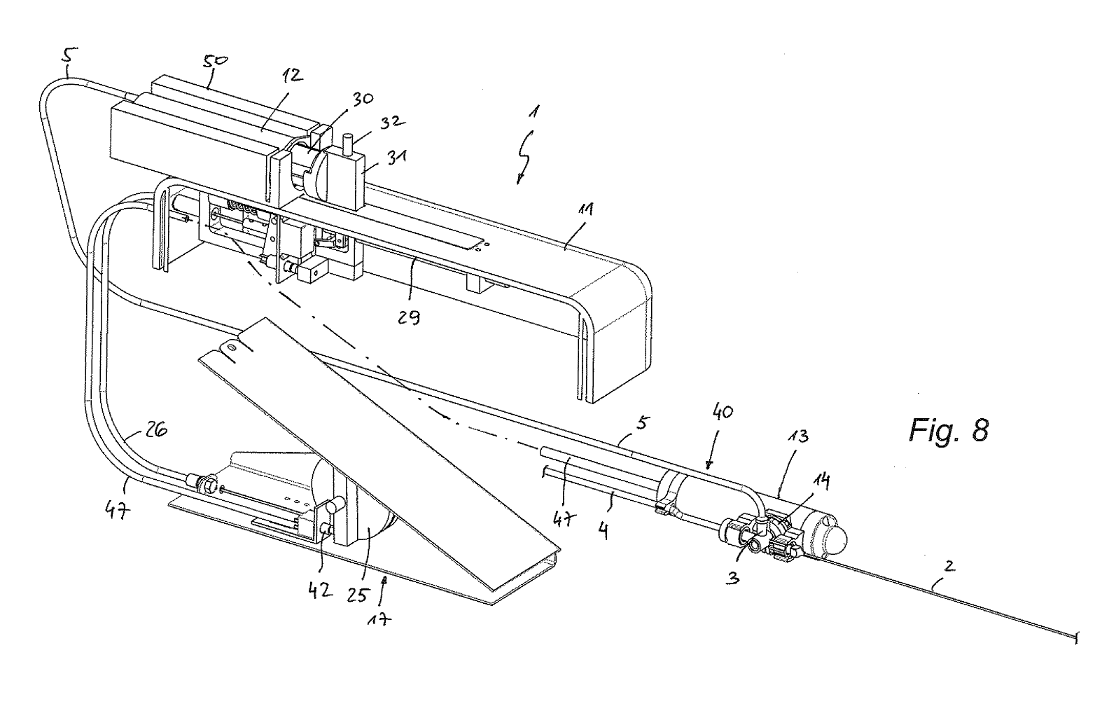

FIG. 8 a second variant of the puncturing device of the invention for extracting egg cells in a three-dimensional partially cut view;

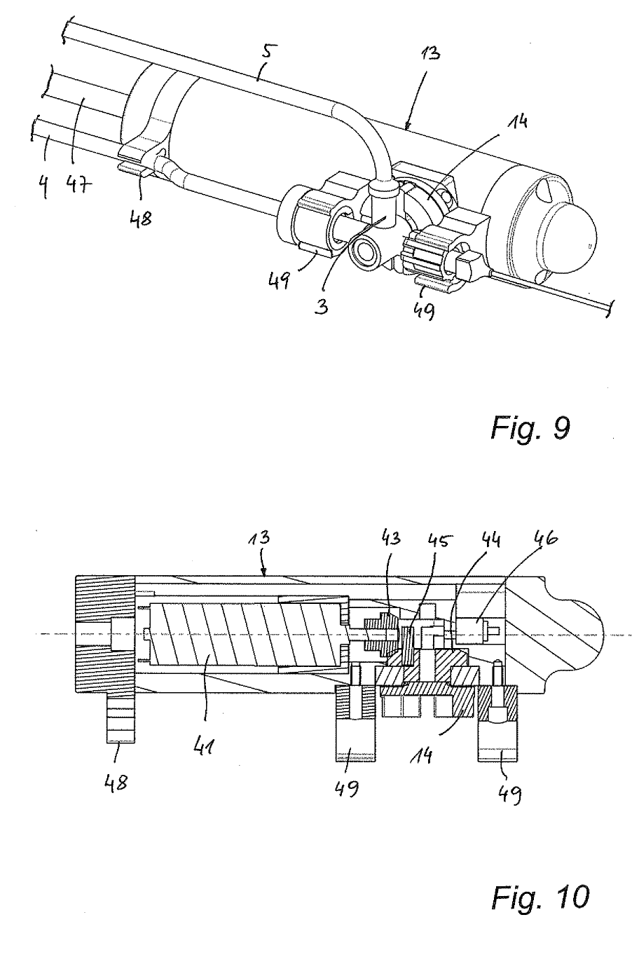

FIG. 9 an enlarged view of the handpiece of the drive mechanism of the multiway valve of FIG. 8; and in

FIG. 10 a sectional view of the handpiece according to FIG. 9 with the multiway valve removed.

The overall view presented in FIG. 1 shows a first variant of the puncturing device 1 for the extraction of egg cells for in-vitro fertilization. The puncturing device 1 comprises a puncturing cannula 2, which can optionally be connected by means of a multiway valve 3 to an aspiration or suction line 4 and a flushing line 5. The multiway valve 3 is connected to a foot-operated drive mechanism 10, by means of which the valve 3 may be switched forth and back between a suction position and a flushing position.

The puncturing cannula 2 has a sharpened needle tip 6 and is connected at the proximal end to the multiway valve 3 via a Luer connection, the suction line 4 leading to a container 7 for the egg cell, respectively the egg-cumulus complex contained in the extracted sample. The container 7 is provided with a connection line 8 to a vacuum source, for instance an electrically powered membrane pump M, which generates the negative pressure required in the lumen 9 of the puncturing cannula 2 for sample extraction.

The flushing line 5 configured as a flexible tube is connected to a container 12 (here shown as a plunger syringe) for the flushing medium, which is attached to a holder 11 (further details are shown in FIG. 7).

FIG. 2 presents an overall view of the essential components of the drive mechanism of the multiway valve 3 as proposed by the invention. The mechanical drive mechanism 10 comprises a handpiece 13 with an actuator part 14, which may be snapped or locked onto the multiway valve 3. To actuate the valve the actuator part 14 is force-connected via a control cable 15 to an actuating element 16 (see FIG. 5) in a foot-operated rocker 17.

As shown in FIGS. 3 and 4 a spring element 18, which can be pre-stressed by the foot-operated rocker 17, is positioned in the handpiece 13 and acts via a toothed rod 19 held in the handpiece 13 on the actuator part 14 actuating the multiway valve 3 in forward or backward direction.

A first actuating element 16 of the foot-operated rocker 17 is provided with a locking element 21 cooperating with a control disk 20, which initiates switching of the multiway valve 3 from suction position to flushing position when the rocker 17 is actuated and turns through a small angle of 3° to 10° departing from the initial position.

The foot-operated rocker 17 comprises a further actuating element 25—as already described in AT 505.749—which is connected via a control cable 26 to a holder 11 holding a plunger syringe connected to the flushing line 5.

The surgeon picks up the puncturing device by the handpiece 13 and inserts the puncturing needle 2 into a follicle. Then he starts up the membrane pump M by means of a foot switch (not shown) and monitors the collapse of the follicle via the ultrasound monitor. The foot-operated rocker 17 is in the initial position and the multiway valve 3 is in suction position.

To initiate the flushing process the surgeon steps on the foot-operated rocker 17, at the beginning of whose motion the multiway valve 3 is switched through 90° to flushing position with the help of the prestressed spring element 18. As the rocker 17 is turned further flushing medium from the plunger syringe 12 can be delivered in the amount required by the surgeon with the help of the force transmitted via the second control cable element 26. When the rocker 17 returns to its initial position, the multiway valve 3 is switched from flushing position to suction position by the control cable element 15 and the spring element 18 is again pre-stressed. Now the surgeon activates a switch or pedal with his other foot to start the membrane pump M and again aspirates the follicle. By means of this procedure the suction/flushing cycle may be carried out repeatedly within a short period of time.

A further advantage of the invention lies in the fact that the handpiece 13 is made of two parts, the first part 22 essentially comprising the actuator part 14 and the toothed rod 19, while the second part 23 can be inserted into the first part 22 and holds the end of the control cable element 15. Both parts 22 and 23 of the handpiece are joined by a spring-loaded locking element 24, which sits in the first part 22 and can be manually released by pressing a key 27. The front part of the handpiece may thus be swiftly detached from the device for sterilization.

As shown in FIG. 7 the foot-operated rocker 17 may be force-connected to the holder 11 of the flushing fluid container 12 configured as a plunger syringe, by means of a control cable element 26 (or a flexible drive shaft). The plunger syringe 12 can be snapped in place into the receiving element 28 of the holder and can thus be easily exchanged.

The cable of the control cable element 26 is attached to a push-rod 29 slideably borne in the holder 11, which moves an actuator element 31 acting on the plunger 30 of the syringe 12 in the direction of the plunger of the plunger syringe 12.

The actuator element 31 has a locking member 32, which establishes a force-locked connection with the push-rod 29 when the foot-operated rocker 17 is pressed in the direction of arrow 33, and which releases said connection when the push-rod driven by the spring 34 moves in the opposite direction. The locking member 32 is loaded by a spring 35 and meshes with the toothing of the push-rod 29 in the manner of a pawl. The locking member 32 may furthermore be manually disengaged (by pressing a finger against the part protruding from the actuator element 31), whereupon the actuator element 31 may be slid backwards on the push-rod 29 in order to replace the plunger syringe 12 by a new one filled with flushing fluid.

In the second variant of the invention shown in FIGS. 8 to 10 the parts are referenced by the same numerals as the corresponding parts of the first variant.

In this alternative variant an electromotive drive 40 is provided for switching the valve 3 from a suction to a flushing position and vice versa. The actuator part 14 is driven by a gear or step motor 41 located in the handpiece 13, which is connected to a switching element 42 in the foot-operated rocker 17.

The holder 11 for the flushing fluid container 12 and the foot-operated rocker 17 are shown partially cut in FIG. 8 for better explanation of their functioning. The flushing fluid container 12 is placed in a heatable or thermostatted holder 50 for keeping the flushing fluid at body temperature.

As described for the first variant, the surgeon inserts the puncturing needle in a follicle and monitors the collapse of the follicle on the ultrasound monitor. The foot-operated rocker 17 is in initial position and the multiway valve 3 is in suction position.

In order to initiate the flushing process the surgeon steps on the rocker 17, whereupon at the beginning of motion the switching element 42 is operated, which actuates the gear motor 41 in the handpiece 13 via a connecting cable 47 and thus turns the multiway valve 3 through 90° to flushing position. By further turning the rocker 17 the amount of flushing fluid required by the surgeon can be supplied from the plunger syringe 12 with the help of force transmission via the control cable element 26. When the rocker 17 returns to its initial state the multiway valve 3 is switched back from flushing to suction position. Now the surgeon activates a switch or pedal (not shown) with his other foot to start the membrane pump M (see FIG. 1) and again aspirates the follicle. By means of this procedure the suction/flushing cycle may be carried out repeatedly within a short period of time.

Via a conical gear 43 the gear motor 41 drives a control disk 44 attached to the actuator part 14, which controls the switching process between suction and flushing position with the help of control pins 45 and terminal switches 46. In the cutting plane as depicted by FIG. 10 only one control pin 45 is to be seen on the control disk 44 and only one terminal switch 46 in the handpiece 13.

To detachably hold the multiway valve 3 together with the puncturing cannula 2 and the suction and flushing lines 4, 5 there are provided on the handpiece 13 locking elements 48, 49 into which the multiway valve 3 plus fittings may be snapped (see FIG. 9).

1-10. (canceled)

11. A puncturing device for extraction of egg cells for in-vitro fertilization (IVF), with a puncturing cannula, which is optionally connectable to a suction line or to a flushing line by means of a multiway valve, wherein the multiway valve is provided with a foot-operated, mechanical or electro-motive drive mechanism for switching the multiway valve from a suction position to a flushing position and vice versa.

12. A puncturing device according to claim 11, wherein the drive mechanism comprises a handpiece with an actuator part, said handpiece being mechanically attachable to the multiway valve carrying the puncturing cannula, using a locking or snap-on connection.

13. A puncturing device according to claim 12, wherein the actuator part for the multiway valve is force-connected via a cable element to an actuator element in a foot-operated rocker.

14. A puncturing device according to claim 13, wherein in the handpiece there is located a spring element prestressed via the foot-operated rocker, which acts on the actuator part through a toothed rod borne in the handpiece to switch the multiway valve.

15. A puncturing device according to claim 13, wherein the actuator element of the foot-operated rocker has a locking element cooperating with a control disk, which locking element initiates a switching process of the multiway valve from suction position to flushing position when the foot-operated rocker is turned through a small angle of 3° to 10°.

16. A puncturing device according to claim 13, wherein the handpiece comprises two parts a first part bearing the actuator part and the toothed rod, and a second part inserted into the first part, which receives the end of the cable element.

17. A puncturing device according to claim 16, wherein the two parts of the handpiece are joined by means of a spring-loaded locking element.

18. A puncturing device according to claim 13, wherein the foot-operated rocker has yet another actuating element, which is connected via a cable element to a holder, which is provided with a plunger syringe connected to the flushing line.

19. A puncturing device according to claim 12, wherein the actuator part for the multiway valve is driven by a step motor placed in the handpiece, which step motor is connected to a switching element in the foot-operated rocker.

20. A puncturing device according to claim 18, wherein the step motor drives via a conical gear a control disk attached to the actuator part, which control disk governs the switching processes between suction and flushing position with the help of control pins and terminal switches.
